(19) United States
(12) Patent Application Publication
(10) Pub. No.: US 2025/0051199 A1
(43) Pub. Date: Feb. 13, 2025

(54) RAPIDLY DISINTEGRATING BUOYANT COMPOSITION

(71) Applicant: BIO-LAB, INC., Lawrenceville, GA (US)

(72) Inventors: Curtis Sayre, Atlanta, GA (US); David Rouse, Smyrna, GA (US); Brian Trenck, Atlanta, GA (US)

(21) Appl. No.: 18/722,060

(22) PCT Filed: Dec. 19, 2022
(86) PCT No.: PCT/US2022/081904

§ 371 Date: Jun. 20, 2024

Related U.S. Application Data
(60) Provisional application No. 63/293,275, filed on Dec. 23, 2021.

(51) Int. Cl. C02F 1/68; C02F 1/50; C02F 1/66; C02F 103/42

(52) U.S. Cl. CPC C02F 1/688; C02F 1/50; C02F 1/66; C02F 2103/42

(57) ABSTRACT

Disclosed herein is a rapidly disintegrating buoyant composition, a process of manufacturing, and a method of use.

### RELATED APPLICATION

This application is being filed on 19 Dec. 2022, as a PCT International patent application, and claims priority to U.S. Provisional Patent Application No. 63/293,275, filed on 23 Dec. 2021, the disclosure of which is hereby incorporated by reference herein in its entirety.

### FIELD

Disclosed herein is a rapidly disintegrating buoyant composition, a process of manufacturing, and a method of use.

### BACKGROUND

Consumers may encounter difficulties when using current chemical packages for dosing water treatment chemicals into their pools/spas. One such difficulty relates to estimating the amount of chemical treatment to apply to a large body of recreational water, which may be due to vagaries of measurement, such as ounces marked on a label, to an incorrect assumption of water volume. Knowing the proper amount needed to dose the proper amount of chemical is crucial to maintaining a safe and aesthetically pleasing pool. There is great interest in providing consumers a simple “unit dose” application of pool chemicals, where there is no measuring needed. They use one dose up to the treatment size, e.g., 2 from 1+X to 2X treatment volumes, etc. This vastly simplifies dosing chemicals.

But unit doses are not the only issue solved by the compositions disclosed herein. Compared to other areas of water treatment by consumers, such as dish washers or washing machines, swimming pool water has a lower degree of agitation. Improper application of a pool chemical can result in the pool chemical settling to the bottom of a pool, where it will quite possibly sit for minutes, hours, or even days before it completely dissolves, resulting in staining or damage to the pool surface. Speed of dissolution is also required for unit dose applications.

Tablets can be made readily via compaction that can accomplish this unit dose application, such as a chlorine dispensing tablet fed via skimmer, feeder, or floater. However, applying these tablets directly to the pool's main body of water would result in their sinking to the bottom of the pool where they would remain for days, slowly damaging and bleaching the pool floor. For instance, tableted products generally have a density (from 1.3 g/cm³ to 2.1 g/cm³) that is greater than water, which means that the tableted product would settle to the bottom of a pool. See, e.g., Yeoman and Connelly. Ideally, the tablet would remain floating when applied to the pool so that it would not contact pool surfaces, thus avoiding damage. Also, by remaining on the surface, any material that sloughed off the tablet would have time/agitation to dissolve as it slowly fell through the column of water after breaking off the tablet.

Thus, a unit dose, floating, rapidly disintegrating tablet would alleviate many problems that previous attempts have failed to provide.

There have been several iterations of pool chemicals in water soluble films as unit dose concepts.

For instance, marketed clarifier products include chitosan in a water-soluble pouch. These products were small pods, as not much chitosan was required to be dosed. But once the chitosan got wet, if the film did not burst just right, the chitosan would clump together and slowly settle in a lump to the floor of the pool.

Additionally, marketed products relate to dissolvable pouches that include pool chemicals. These products were much larger pods of pool chemicals, up to 8 ounces. Several of the chemicals in the pouches, such as a dichlor-based shock and sodium bisulfate, were incompatible with the film, causing the film to become brittle and break before dosing. Other chemicals, such as sodium carbonate, would fuse into a solid mass once the film ruptured in water. This mass of sodium carbonate would either require days on the pool bottom to dissolve or would require force, such as chopping it up into smaller chunks, to get it to dissolve faster.

In 2013, BioLab launched the “Angry Egg” that required a plastic shell around effervescing tablets as a unit dose device. Sayre. The product would sink upon introduction to the pool, where the “egg” shape around the chemicals would prevent their contact of the pool surface. Once the material inside the egg dissolved, the “shell” would slowly float to the surface to be removed. The chemicals within the shell may not have completely dissolved before the buoyancy of the shell lifted them to the surface.

To minimize damage to pool surfaces, development activity focused on a rapidly disintegrating buoyant composition (e.g., tablet). A buoyant composition has the advantage of rapid disintegration without contacting a pool surface, and thus, no damage occurs. Further, the effervescence, while visually compelling, creates agitation/fragmentation which dissolves the tablets in several minutes with the gas generation providing the buoyancy to keep the tablet afloat. The technology is compatible amongst itself, does not require application packaging and will not negatively impact pool chemistry.

### SUMMARY

Disclosed herein is a rapidly disintegrating buoyant composition, a process of manufacturing, and a method of use.

### DETAILED DESCRIPTION

The information that follows illustrates various embodiments of the compositions disclosed herein. For the avoidance of doubt, it is specifically intended that any particular feature(s) described individually in any one of these paragraphs (or part thereof) may be combined with one or more other features described in one or more of the remaining paragraphs (or part thereof). In other words, it is explicitly intended that the features described below individually in each paragraph (or part thereof) represent aspects of the invention that may be taken in isolation and also combined with other aspects of the composition described elsewhere within this specification as a whole and including the examples, as well as a process for preparing the composition and a method of using the composition. The skilled person will appreciate that the compositions claimed herein extends to such combinations of features and that these have not been recited in detail here in the interests of brevity.

Definitions of some of the terms used herein are detailed below.

The use of the terms “a” and “an” and “the” and similar references in the context of describing the liquid composition described herein (especially in the context of the following claims) are to be construed to cover both the singular and the plural, unless otherwise indicated herein or clearly contradicted by context.

The term “about” as used herein embodies standard error associated with a physico-chemical observable. As used herein, the term “about” means a slight variation of the value specified, for example, within 10% of the value specified. A stated amount for a compositional ingredient that is not preceded by the term “about” does not mean that there is no variance for the stated term, as one of ordinary skill would understand that there may be the possibility of a degree of variability generally associated with experimental error.

Certain compositions disclosed herein may include one or more ingredients that may expressed in percent by weight of ingredient based on the total weight of the composition (viz., % w/w) or in percent by volume of the ingredient based on the total volume of the composition (viz., % v/v).

A first embodiment relates to a rapidly disintegrating buoyant composition, comprising: a) an active agent, b) at least about 15% w/w of an organic acid, c) at least about 10% w/w of a carbonate, and d) about 1 to about 30% w/w of a skin oil, a fragrance, a surfactant, an adjuvant, or a combination thereof.

An active agent (or payload) may comprise a clarifier, an algicide, a phosphate removal composition, a surfactant, an enzyme, a purifying agent, a scale reducing agent, a flocculant, a chelating agent, alkalinity stabilizing agent, non-chlorine oxidant, a fragrance, a dye, a water stabilizer/conditioner, a calcium hardness adjusting agent, a buffering agent, a CYA removal agent, a chlorine and/or chloramine reducing agent, a pH decreasing agent (e.g., an organic acid), a pH increasing agent (e.g., a carbonate), or a combination thereof, examples of which are summarized below.
A clarifier may comprise at least one aluminum sulfate (e.g., Al₂ (SO₄)₃·nH₂O), MAl(SO₄)₂·nH₂O, where M=Na⁺, K⁺, NH₄⁺, etc., n=0-12, e.g., sodium aluminum sulfate (e.g., NaAl(SO₄)₂, NaAl(SO₄)₂·.12H₂O, etc.), potassium aluminum sulfate (e.g., KAl(SO₄)₂, KAl(SO₄)₂·12H₂O, etc.), ammonium aluminum sulfate (e.g., NH₄Al(SO₄)₂, NH₄Al(SO₄)₂·12H₂O, etc.). Alternatively a clarifier may comprise a chitosan or a salt thereof, a diallyldimethylammonium chloride homopolymer (e.g., polyDADMAC (CAS No. 26062-79-3)), a diallyldimethylammonium chloride (“DADMAC”) containing polymer (e.g., CAS Nos. 153891-25-9, 885610-94-6, 153891-21-5, 217643-64-6, 374900-36-4, etc.), and the like. Yet an alternative clarifier may comprise sodium hexametaphosphate (SHMP, e.g., Na₆[(PO₃)₆], CAS No. 68915-31-1);
An algicide may comprise a copper sulfate (e.g., CuSO₄·nH₂O, where n=0-5, e.g., CuSO₄·5H₂O), a copper citrate (e.g., Cu₃(C₆H₅O₇)₂), a quaternary ammonium compound (e.g., an alkylbenzyldimethyl ammonium chloride (e.g., ADBAC, CAS No. 8001-54-5)), and the like;
A phosphate removal composition may comprise a lanthanum chloride (e.g., LaCl₃, LaCl₃·7H₂O, etc.), a lanthanum carbonate (e.g., La₂(CO₃)₂), a lanthanum sulfate (La₂(SO₄)₃ (CAS No. 10099-60-2) or a hydrate thereof), an aluminum sulfate, a zirconyl chloride (e.g., ZrOCl₂·8H₂O), or a combination thereof;
a surfactant may be anionic, cationic, a non-ionionic, a zwitterionic surfactant, and/or an N-oxide surfactant, for example, the surfactant may be non-ionic, for example, a polyethylene glycol undecyl ether (e.g., a non-ionic surfactant marketed as Tomadol® (e.g., Tomadol 1-3, Tomadol 1-5, Tomadol 1-6, Tomadol 1-7, Tomadol 1-9 (CAS No. 34398-01-1), etc.): an α-hydro-ω-hydroxypoly(oxyethylene)poly(oxypropylene)poly(oxyethylene) block copolymer (aka Poloxamer or Pluronic, e.g., Poloxamer 124, Poloxamer 188, Poloxamer 237, etc.): a C8-C10 alkyl polyglycoside (e.g., Glucopon® 215, Glucopon® 225, Glucopon® 425, Glucopon® 425 N, and the like, see, e.g., CAS No. 193226-14-1); an α-(nonylphenyl)-ω-hydroxy-poly(oxy-1,2-ethanediyl) (e.g., Makon®4, Makon®) 6, Makon® 8, Makon® 10, Makon® 12, Makon® 14, etc., see, e.g., CAS No. 9016-45-9): a polyoxyethylene sorbitan fatty acid ester (generally referred to as Polysorbate or “Tween,” e.g., polyoxyethylene 20 sorbitan monolaurate (Polysorbate 20 (or Tween 20), CAS No. 6005-64-5), polyoxyethylene 40 sorbitan monopalmitate (Polysorbate 40 (or Tween 40), CAS No. 9005-66-7, as well as Polysorbate 60 (CAS No. 9005-67-8), Polysorbate 80 (CAS No. 9005-65-6), Polysorbate 120 (CAS No. 66794-58-9), and the like): a polyoxyalkylene glycol oleic acid condensate (manufactured by Bio-Soft, see, e.g., CAS Nos. 140881-29-4, 140881-27-2, 140881-28-3, 140880-97-3, 140880-96-2, etc.); and the like; or a combination thereof;
An enzyme may comprise a protease, an amylase, a lipase, a cellulase, a mannanase, oxidoreductases, transferases, hydrolases, lyases, isomerases, ligases, and translocases, and the like, or a combination thereof;
A purifying agent, e.g., sodium iodide (NaI (CAS No. 7681-82-5)), potassium iodide (KI (CAS No. 7681-11-0)), sodium iodate (NaIO₃ (CAS No. 7681-55-2)), potassium iodate (KIO₃ (CAS No. 7758 May 6)), sodium metaperiodate (NaIO₄ (CAS No. 7790-28-5)), potassium metaperiodate (KIO₄ (CAS No. 7790-21-8)), and the like, or a combination thereof;
Stain and/or scale reducing agent, e.g., 2-phosphono-1,2,4-butanetricarboxylic acid (PBTC (CAS No. 37971-36-1)) or a salt thereof: P,P′-(1-hydroxyethylidene)bis-phosphonic acid (HEDP (CAS No. 2809-21-4)) or a salt thereof (e.g., HEDP sodium salt), ascorbic acid or a salt thereof (used, optionally, in combination with an enzyme), oxalic acid (e.g., C₂H₂O₄ (CAS No. 144-62-7) or a salt thereof, polyphosphate, a neutralized phosphonocarboxylic acid (e.g., ALBRITE 300 (CAS 180513-31-9, a reaction product of phosphonic acid (or a sodium salt thereof) with maleic anhydride), a polymer, or a combination thereof;
Flocculant, e.g., poly aluminum chloride (e.g., CAS No. 1327-41-9), aluminum chlorohydrate (e.g., Al₂(OH)₅Cl·2H₂O), and the like;
Chelating agent (e.g., ethylene diamine tetraacetic acid (EDTA (CAS No. 60-00-4)) or a salt thereof (e.g., disodium EDTA, dipotassium EDTA, etc.), and the like;
Alkalinity stabilizing agent, e.g., sodium tetraborate (e.g., Na₂B₄O₇ (CAS No. 1330-43-4)), boric acid (e.g., B(OH)₃ (CAS No. 10043-35-3)), and the like;
A non-chlorine oxidant, e.g., oxone (Potassium peroxymonosulfate sulfate, e.g., 2KHSO₅·KHSO₄·K₂SO₄ (CAS Nos. 37222-66-5 and 70693-62-8)), sodium percarbonate (e.g., Na₂C₂O₅ (CAS No. 3313-92-6)), Na₂CO₄ (CAS No. 4452-58-8), Na₂CO₃·1.5H₂O₂ (CAS No. 15630-89-4), sodium perborate (e.g., NaBO₃ (CAS Nos. 11138-47-9 and 7632-04-4));
A fragrance includes any suitable fragrance, for example, a fragrance in a liquid (e.g., oil-based) or solid (wax-based) vehicle, e.g., Starburst, rose, peppermint, lavender, sandalwood, chamomile, tea tree, jasmine, and the like.
A dye (i.e. a colorant bomb), includes a stain-free colorant of red, green, blue, and the like;
Water stabilizer/conditioner (e.g., cyanuric acid (C₃H₃N₃O₃, “CYA,” CAS No. 108-80-5)), or a salt thereof).
Calcium hardness adjusting agent, e.g., calcium chloride (e.g., CaCl₂) (CAS No. 10043-52-4)) and/or calcium carbonate (e.g., CaCO₃ (CAS No. 471-34-1));
Buffering agent, e.g., sodium salts of phosphoric acid (e.g., sodium phosphate monobasic (e.g., NaH₂PO₄ (CAS No. 7558-80-7), sodium phosphate dibasic (e.g., Na₂HPO₄ (CAS No. 7558-79-4), sodium phosphate tribasic (e.g., Na₃PO₄ (CAS No. 7601-54-9));
A CYA removal agent, e.g., an active bacterial culture capable of reducing CYA content; and
A chlorine and/or chloramine reducing agent (e.g., sodium sulfite, sodium thiosulfate).

In one aspect of the first embodiment, the organic acid may be present in an amount of at least 20% w/w of an organic acid and the carbonate may be present in an amount of at least 10% w/w of a carbonate.

A suitable organic acid includes an acid comprising a carboxylic acid moiety that exists as a solid at room temperature. Examples of organic acids include, but are not limited to, aconitic acid, adipic acid, ascorbic acid, benzoic acid, citric acid, gluconic acid, glutaric acid, glyceric acid, lactic acid, malic acid, malonic acid, succinic acid, tartaric acid, or a combination thereof.

In one aspect, the organic acid comprises adipic acid, benzoic acid, citric acid, malonic acid, succinic acid, tartaric acid, or a combination thereof.

In another aspect, the organic acid comprises citric acid or a hydrate thereof (e.g., citric acid monohydrate).

A suitable carbonate includes a bicarbonate, a carbonate, a sesquicarbonate, or a combination thereof. Examples of suitable carbonates include, but are not limited to, sodium bicarbonate, sodium carbonate, sodium sesquicarbonate, potassium bicarbonate, potassium carbonate, potassium sesquicarbonate, and the like.

In another aspect, the carbonate comprises sodium carbonate or a hydrate thereof (e.g., sodium carbonate monohydrate).

In one aspect, the rapidly disintegrating buoyant composition has an equivalent ratio of the organic acid to the carbonate that ranges from about 0.3 to about 1.1, and all values in between, including for example, about 0.4, about 0.5, about 0.6, about 0.7, about 0.8, about 0.9, and about 1.0.

In yet another aspect, the rapidly disintegrating buoyant composition has an equivalent ratio of the organic acid to the carbonate of about 0.6 to about 1.1 and all values in between, including for example, about 0.65, about 0.70, about 0.75, about 0.80, about 0.85, about 0.90, about 0.95, about 1.00, and about 1.05.

In certain instances (e.g., composition that increases/decreases pH), the equivalent ratio of the organic acid to the carbonate may exceed the stated bounds of about 0.3 to about 1.1.

One will appreciate that the equivalent weight (g/Eq) of an organic acid may be obtained by dividing the molecular weight (g/mol) by the number of acid equivalents of the organic acid. Further, one will appreciate that the equivalent weight (g/Eq) of a carbonate may be obtained by dividing the molecular weight (g/mol) by the number of basic equivalents of the carbonate.

For convenience, the following table identifies exemplary organic acids (or a hydrate thereof) and carbonates (or a hydrate thereof), their molecular weights (“MW,” g/mol), the number of acid/basic equivalents (Eq.), and the equivalent weights (“EW,” g/Eq.), where the hydrate may be a monohydrate (“MH”), a dihydrate (“DH”), or a decahydrate (“DcH”), among others.

| Organic Acid/Carbonate | MW (g/mol) | Eq. | EW (g/Eq.) |
|---|---|---|---|
| Aconitic acid | 174.108 | 3 | 58.04 |
| Adipic acid | 146.14 | 2 | 73.07 |
| Ascorbic acid | 176.13 | 2 | 88.065 |
| Benzoic acid | 122.12 | 1 | 122.12 |
| Citric acid | 192.124 | 3 | 64.04 |
| Citric acid MH | 210.14 | 3 | 70.05 |
| Gluconic acid | 196.16 | 2 | 98.08 |
| Glutaric acid | 132.12 | 2 | 66.06 |
| Glyceric acid | 106.08 | 1 | 106.08 |
| Lactic acid | 90.08 | 1 | 90.08 |
| Malic acid | 134.09 | 2 | 67.05 |
| Malonic acid | 104.062 | 2 | 52.03 |
| Succinic acid | 118.09 | 2 | 59.05 |
| Tartaric acid | 150.09 | 2 | 75.05 |
| Sodium bicarbonate | 84.01 | 1 | 84.01 |
| Sodium carbonate | 105.99 | 2 | 53.0 |
| Sodium carbonate MH | 124.0 | 2 | 62.0 |
| Sodium carbonate DH | 286.1 | 2 | 143.05 |
| Sodium sesquicarbonate | 189.99 | 4 | 47.50 |
| Sodium sesquicarboate DcH | 226.03 | 4 | 56.51 |

To illustrate the concept of the equivalent ratio of the organic acid to the carbonate that ranges from about 0.3 to about 1.1, it is instructive to consider illustrative calculations. For instance, a composition that includes about 100 g of citric acid monohydrate and about 250 g of sodium carbonate has an equivalent ratio of citric acid to carbonate of about 0.3, where about 100 g citric acid monohydrate includes about 1.43 Eq. citric acid (100 g/70.05 g/Eq) and where about 250 g sodium carbonate includes about 4.7 Eq. of carbonate (250 g/53.0 g/Eq). As another example, a composition that includes about 200 g citric acid monohydrate and about 250 g sodium carbonate has an equivalent ratio of citric acid to carbonate of about 0.6, where about 200 g citric acid monohydrate includes about 2.86 Eq. citric acid (200 g/70.05 g/Eq) and where about 250 g sodium carbonate includes about 4.7 Eq. of carbonate (250 g/53.0 g/Eq).

Compositions contemplated herein comprise at least about 15% w/w of an organic acid. In one aspect, the composition comprises from about 15% w/w to about 75% w/w of an organic acid.

In yet another aspect, compositions contemplated herein comprise from about 15% w/w to about 75% w/w of an organic acid, including all values in between, for example, about 16% w/w, about 17% w/w, about 18% w/w, about 19% w/w; about 20% w/w; about 20.1% w/w; about 20.2% w/w, about 20.3% w/w, about 20.4% w/w; about 20.5% w/w; about 20.6% w/w; about 20.7% w/w; about 20.8% w/w; about 20.9% w/w, about 21.0% w/w; about 21.1% w/w, about 21.2% w/w, about 21.3% w/w; about 21.4% w/w; about 21.5% w/w; about 21.6% w/w; about 21.7% w/w, about 21.8% w/w, about 21.9% w/w; about 22.0% w/w; about 22.1% w/w; about 22.2% w/w, about 22.3% w/w; about 22.4% w/w, about 22.5% w/w; about 22.6% w/w, about 22.7% w/w; about 22.8% w/w; about 22.9% w/w; about 23.0% w/w, about 23.1% w/w; about 23.2% w/w; about 23.3% w/w; about 23.4% w/w, about 23.5% w/w; about 23.6% w/w; about 23.7% w/w, about 23.8% w/w, about 23.9% w/w, about 24.0% w/w, about 24.1% w/w, about 24.2% w/w; about 24.3% w/w, about 24.4% w/w, about 24.5% w/w, about 24.6% w/w; about 24.7% w/w; about 24.8% w/w; about 24.9% w/w, about 25.0% w/w, 25.1% w/w, about 25.2% w/w; about 25.3% w/w, about 25.4% w/w, about 25.5% w/w, about 25.6% w/w, about 25.7% w/w; about 25.8% w/w, about 25.9% w/w, 26.1% w/w; about 26.2% w/w, about 26.3% w/w; about 26.4% w/w, about 26.5% w/w, about 26.6% w/w, about 26.7% w/w; about 26.8% w/w, about 26.9% w/w; about 26.0% w/w, 27.1% w/w, about 27.2% w/w; about 27.3% w/w, about 27.4% w/w, about 27.5% w/w, about 27.6% w/w, about 27.7% w/w; about 27.8% w/w, about 27.9% w/w, about 27.0% w/w, 28.1% w/w; about 28.2% w/w; about 28.3% w/w, about 28.4% w/w, about 28.5% w/w, about 28.6% w/w, about 28.7% w/w, about 28.8% w/w, about 28.9% w/w, about 28.0% w/w, 29.1% w/w, about 29.2% w/w, about 29.3% w/w, about 29.4% w/w, about 29.5% w/w, about 29.6% w/w, about 29.7% w/w, about 29.8% w/w, about 29.9% w/w, about 30.0% w/w, 30.1% w/w, about 30.2% w/w, about 30.3% w/w, about 30.4% w/w, about 30.5% w/w, about 30.6% w/w, about 30.7% w/w, about 30.8% w/w, about 30.9% w/w, about 31.0% w/w, 31.1% w/w, about 31.2% w/w, about 31.3% w/w, about 31.4% w/w, about 31.5% w/w, about 31.6% w/w, about 31.7% w/w, about 31.8% w/w, about 31.9% w/w, about 32.0% w/w, 32.1% w/w, about 32.2% w/w, about 32.3% w/w, about 32.4% w/w, about 32.5% w/w, about 32.6% w/w, about 32.7% w/w, about 32.8% w/w, about 32.9% w/w, about 33.0% w/w, 33.1% w/w, about 33.2% w/w, about 33.3% w/w, about 33.4% w/w, about 33.5% w/w, about 33.6% w/w, about 33.7% w/w, about 33.8% w/w, about 33.9% w/w, about 34.0% w/w, 34.1% w/w, about 34.2% w/w, about 34.3% w/w, about 34.4% w/w, about 34.5% w/w, about 34.6% w/w, about 34.7% w/w, about 34.8% w/w, about 34.9% w/w, about 35.0% w/w, 35.1% w/w, about 35.2% w/w, about 35.3% w/w, about 35.4% w/w, about 35.5% w/w, about 35.6% w/w, about 35.7% w/w, about 35.8% w/w, about 35.9% w/w, about 36.0% w/w, 36.1% w/w, about 36.2% w/w, about 36.3% w/w, about 36.4% w/w, about 36.5% w/w, about 36.6% w/w, about 36.7% w/w, about 36.8% w/w, about 36.9% w/w, about 37.0% w/w, 37.1% w/w, about 37.2% w/w, about 37.3% w/w, about 37.4% w/w, about 37.5% w/w, about 37.6% w/w, about 37.7% w/w, about 37.8% w/w, about 37.9% w/w, about 38.0% w/w, 38.1% w/w, about 38.2% w/w, about 38.3% w/w, about 38.4% w/w, about 38.5% w/w, about 38.6% w/w, about 38.7% w/w, about 38.8% w/w, about 38.9% w/w, about 39.0% w/w, 39.1% w/w, about 39.2% w/w, about 39.3% w/w, about 39.4% w/w, about 39.5% w/w, about 39.6% w/w, about 39.7% w/w, about 39.8% w/w, about 39.9% w/w, about 40.0% w/w, 40.1% w/w, about 40.2% w/w, about 40.3% w/w, about 40.4% w/w, about 40.5% w/w, about 40.6% w/w, about 40.7% w/w, about 40.8% w/w, about 40.9% w/w, about 41.0% w/w, 41.1% w/w, about 41.2% w/w, about 41.3% w/w, about 41.4% w/w, about 41.5% w/w, about 41.6% w/w, about 41.7% w/w, about 41.8% w/w, about 41.9% w/w, about 42.0% w/w, 42.1% w/w, about 42.2% w/w, about 42.3% w/w, about 42.4% w/w, about 42.5% w/w, about 42.6% w/w, about 42.7% w/w, about 42.8% w/w, about 42.9% w/w, about 43.0% w/w, 43.1% w/w, about 43.2% w/w, about 43.3% w/w, about 43.4% w/w, about 43.5% w/w, about 43.6% w/w, about 43.7% w/w, about 43.8% w/w, about 43.9% w/w, about 44.0% w/w, 44.1% w/w, about 44.2% w/w, about 44.3% w/w, about 44.4% w/w, about 44.5% w/w, about 44.6% w/w, about 44.7% w/w, about 44.8% w/w, about 44.9% w/w, about 45.0% w/w, 45.1% w/w, about 45.2% w/w, about 45.3% w/w, about 45.4% w/w, about 45.5% w/w, about 45.6% w/w, about 45.7% w/w, about 45.8% w/w, about 45.9% w/w, about 46.0% w/w, 46.1% w/w, about 46.2% w/w, about 46.3% w/w, about 46.4% w/w, about 46.5% w/w, about 46.6% w/w, about 46.7% w/w, about 46.8% w/w, about 46.9% w/w, about 47.0% w/w, 47.1% w/w, about 47.2% w/w, about 47.3% w/w, about 47.4% w/w, about 47.5% w/w, about 47.6% w/w, about 47.7% w/w, about 47.8% w/w, about 47.9% w/w, about 48.0% w/w, 48.1% w/w, about 48.2% w/w, about 48.3% w/w, about 48.4% w/w, about 48.5% w/w, about 48.6% w/w, about 48.7% w/w, about 48.8% w/w, about 48.9% w/w, about 49.0% w/w, 49.1% w/w, about 49.2% w/w, about 49.3% w/w, about 49.4% w/w, about 49.5% w/w, about 49.6% w/w, about 49.7% w/w, about 49.8% w/w, about 49.9% w/w, about 50.0% w/w, 50.1% w/w, about 50.2% w/w, about 50.3% w/w, about 50.4% w/w, about 50.5% w/w, about 50.6% w/w, about 50.7% w/w, about 50.8% w/w, about 50.9% w/w, about 51.0% w/w, 51.1% w/w, about 51.2% w/w, about 51.3% w/w, about 51.4% w/w, about 51.5% w/w, about 51.6% w/w, about 51.7% w/w, about 51.8% w/w, about 51.9% w/w, about 52.0% w/w, 52.1% w/w, about 52.2% w/w, about 52.3% w/w, about 52.4% w/w, about 52.5% w/w, about 52.6% w/w, about 52.7% w/w, about 52.8% w/w, about 52.9% w/w, about 53.0% w/w, 53.1% w/w, about 53.2% w/w, about 53.3% w/w, about 53.4% w/w, about 53.5% w/w, about 53.6% w/w, about 53.7% w/w, about 53.8% w/w, about 53.9% w/w, about 54.0% w/w, 54.1% w/w, about 54.2% w/w, about 54.3% w/w, about 54.4% w/w, about 54.5% w/w, about 54.6% w/w, about 54.7% w/w, about 54.8% w/w, about 54.9% w/w, about 55.0% w/w, 55.1% w/w, about 55.2% w/w, about 55.3% w/w, about 55.4% w/w, about 55.5% w/w, about 55.6% w/w, about 55.7% w/w, about 55.8% w/w, about 55.9% w/w, about 56.0% w/w, 56.1% w/w, about 56.2% w/w, about 56.3% w/w, about 56.4% w/w, about 56.5% w/w, about 56.6% w/w, about 56.7% w/w, about 56.8% w/w, about 56.9% w/w, about 57.0% w/w, 57.1% w/w, about 57.2% w/w, about 57.3% w/w, about 57.4% w/w, about 57.5% w/w, about 57.6% w/w, about 57.7% w/w, about 57.8% w/w, about 57.9% w/w, about 58.0% w/w, 58.1% w/w, about 58.2% w/w, about 58.3% w/w, about 58.4% w/w, about 58.5% w/w, about 58.6% w/w, about 58.7% w/w, about 58.8% w/w, about 58.9% w/w, about 59.0% w/w, 59.1% w/w, about 59.2% w/w, about 59.3% w/w, about 59.4% w/w, about 59.5% w/w, about 59.6% w/w, about 59.7% w/w, about 59.8% w/w, about 59.9% w/w, about 60.0% w/w, 60.1% w/w, about 60.2% w/w, about 60.3% w/w, about 60.4% w/w, about 60.5% w/w, about 60.6% w/w, about 60.7% w/w, about 60.8% w/w, about 60.9% w/w, about 61.0% w/w, 61.1% w/w, about 61.2% w/w, about 61.3% w/w, about 61.4% w/w, about 61.5% w/w, about 61.6% w/w, about 61.7% w/w, about 61.8% w/w, about 61.9% w/w, about 62.0% w/w, 62.1% w/w, about 62.2% w/w, about 62.3% w/w, about 62.4% w/w, about 62.5% w/w, about 62.6% w/w, about 62.7% w/w, about 62.8% w/w, about 62.9% w/w, about 63.0% w/w, 63.1% w/w, about 63.2% w/w, about 63.3% w/w, about 63.4% w/w, about 63.5% w/w, about 63.6% w/w, about 63.7% w/w, about 63.8% w/w, about 63.9% w/w, about 64.0% w/w, 64.1% w/w, about 64.2% w/w, about 64.3% w/w, about 64.4% w/w, about 64.5% w/w, about 64.6% w/w, about 64.7% w/w, about 64.8% w/w, about 64.9% w/w, about 65.0% w/w, 65.1% w/w, about 65.2% w/w, about 65.3% w/w, about 65.4% w/w, about 65.5% w/w, about 65.6% w/w, about 65.7% w/w, about 65.8% w/w, about 65.9% w/w, about 66.0% w/w, 66.1% w/w, about 66.2% w/w, about 66.3% w/w, about 66.4% w/w, about 66.5% w/w, about 66.6% w/w, about 66.7% w/w, about 66.8% w/w, about 66.9% w/w, about 67.0% w/w, 67.1% w/w, about 67.2% w/w, about 67.3% w/w, about 67.4% w/w, about 67.5% w/w, about 67.6% w/w, about 67.7% w/w, about 67.8% w/w, about 67.9% w/w, about 68.0% w/w, 68.1% w/w, about 68.2% w/w, about 68.3% w/w, about 68.4% w/w, about 68.5% w/w, about 68.6% w/w, about 68.7% w/w, about 68.8% w/w, about 68.9% w/w, about 69.0% w/w, 69.1% w/w, about 69.2% w/w, about 69.3% w/w, about 69.4% w/w, about 69.5% w/w, about 69.6% w/w, about 69.7% w/w, about 69.8% w/w, about 69.9% w/w, about 70.0% w/w, 70.1% w/w, about 70.2% w/w, about 70.3% w/w, about 70.4% w/w, about 70.5% w/w, about 70.6% w/w, about 70.7% w/w, about 70.8% w/w, about 70.9% w/w, about 71.0% w/w, 71.1% w/w, about 71.2% w/w, about 71.3% w/w, about 71.4% w/w, about 71.5% w/w, about 71.6% w/w, about 71.7% w/w, about 71.8% w/w, about 71.9% w/w, about 72.0% w/w, 72.1% w/w, about 72.2% w/w, about 72.3% w/w, about 72.4% w/w, about 72.5% w/w, about 72.6% w/w, about 72.7% w/w, about 72.8% w/w, about 72.9% w/w, about 73.0% w/w, 73.1% w/w, about 73.2% w/w, about 73.3% w/w, about 73.4% w/w, about 73.5% w/w, about 73.6% w/w, about 73.7% w/w, about 73.8% w/w, about 73.9% w/w, about 74.0% w/w. 74.1% w/w, about 74.2% w/w, about 74.3% w/w, about 74.4% w/w, about 74.5% w/w, about 74.6% w/w, about 74.7% w/w, about 74.8% w/w, and about 74.9% w/w.

In certain aspects, the active agent (or payload) may comprise an organic acid, e.g., a pH reducing composition, where the organic acid serves a dual function-namely, reduce the pH of the recreational water and facilitate disintegration of the composition. Accordingly, another aspect relates to a composition comprising from about 15% w/w to about 95% w/w of an organic acid and all values in between, as indicated above.

Compositions contemplated herein comprise at least about 10% w/w of a carbonate. In one aspect, the composition comprises from about 10% w/w to about 75% w/w of a carbonate.

In yet another aspect, compositions contemplated herein comprise from about 10% w/w to about 75% w/w of a carbonate, including all values in between, for example, about 10.1% w/w, about 10.2% w/w, about 10.3% w/w, about 10.4% w/w, about 10.5% w/w, about 10.6% w/w, about 10.7% w/w, about 10.8% w/w, about 10.9% w/w, about 11.0% w/w, about 11.1% w/w, about 11.2% w/w, about 11.3% w/w, about 11.4% w/w, about 11.5% w/w, about 11.6% w/w, about 11.7% w/w, about 11.8% w/w, about 11.9% w/w, about 12.0% w/w, about 12.1% w/w, about 12.2% w/w, about 12.3% w/w, about 12.4% w/w, about 12.5% w/w, about 12.6% w/w, about 12.7% w/w, about 12.8% w/w, about 12.9% w/w, about 13.0% w/w, about 13.1% w/w, about 13.2% w/w, about 13.3% w/w, about 13.4% w/w, about 13.5% w/w, about 13.6% w/w, about 13.7% w/w, about 13.8% w/w, about 13.9% w/w, about 14.0% w/w, about 14.1% w/w, about 14.2% w/w, about 14.3% w/w, about 14.4% w/w, about 14.5% w/w, about 14.6% w/w, about 14.7% w/w, about 14.8% w/w, about 14.9% w/w, about 15.0% w/w, about 15.1% w/w, about 15.2% w/w, about 15.3% w/w, about 15.4% w/w, about 15.5% w/w, about 15.6% w/w, about 15.7% w/w, about 15.8% w/w, about 15.9% w/w, about 16.0% w/w, about 16.1% w/w, about 16.2% w/w, about 16.3% w/w, about 16.4% w/w, about 16.5% w/w, about 16.6% w/w, about 16.7% w/w, about 16.8% w/w, about 16.9% w/w, about 17.0% w/w, about 17.1% w/w, about 17.2% w/w, about 17.3% w/w, about 17.4% w/w, about 17.5% w/w, about 17.6% w/w, about 17.7% w/w, about 17.8% w/w, about 17.9% w/w, about 18.0% w/w, about 18.1% w/w, about 18.2% w/w, about 18.3% w/w, about 18.4% w/w, about 18.5% w/w, about 18.6% w/w, about 18.7% w/w, about 18.8% w/w, about 18.9% w/w, about 19.0% w/w, about 19.1% w/w, about 19.2% w/w, about 19.3% w/w, about 19.4% w/w, about 19.5% w/w, about 19.6% w/w, about 19.7% w/w, about 19.8% w/w, about 19.9% w/w, about 20.1% w/w, about 20.2% w/w, about 20.3% w/w, about 20.4% w/w, about 20.5% w/w, about 20.6% w/w, about 20.7% w/w, about 20.8% w/w, about 20.9% w/w, about 21.0% w/w, about 21.1% w/w, about 21.2% w/w, about 21.3% w/w, about 21.4% w/w, about 21.5% w/w, about 21.6% w/w, about 21.7% w/w, about 21.8% w/w, about 21.9% w/w, about 22.0% w/w, about 22.1% w/w, about 22.2% w/w, about 22.3% w/w, about 22.4% w/w, about 22.5% w/w, about 22.6% w/w, about 22.7% w/w, about 22.8% w/w, about 22.9% w/w, about 23.0% w/w, about 23.1% w/w, about 23.2% w/w, about 23.3% w/w, about 23.4% w/w, about 23.5% w/w, about 23.6% w/w, about 23.7% w/w, about 23.8% w/w, about 23.9% w/w, about 24.0% w/w, about 24.1% w/w, about 24.2% w/w, about 24.3% w/w, about 24.4% w/w, about 24.5% w/w, about 24.6% w/w, about 24.7% w/w, about 24.8% w/w, about 24.9% w/w, about 25.0% w/w, 25.1% w/w, about 25.2% w/w, about 25.3% w/w, about 25.4% w/w, about 25.5% w/w, about 25.6% w/w, about 25.7% w/w, about 25.8% w/w, about 25.9% w/w, 26.1% w/w, about 26.2% w/w, about 26.3% w/w, about 26.4% w/w, about 26.5% w/w, about 26.6% w/w, about 26.7% w/w, about 26.8% w/w, about 26.9% w/w, about 26.0% w/w, 27.1% w/w, about 27.2% w/w, about 27.3% w/w, about 27.4% w/w, about 27.5% w/w, about 27.6% w/w, about 27.7% w/w, about 27.8% w/w, about 27.9% w/w, about 27.0% w/w, 28.1% w/w, about 28.2% w/w, about 28.3% w/w, about 28.4% w/w, about 28.5% w/w, about 28.6% w/w, about 28.7% w/w, about 28.8% w/w, about 28.9% w/w, about 28.0% w/w, 29.1% w/w, about 29.2% w/w, about 29.3% w/w, about 29.4% w/w, about 29.5% w/w, about 29.6% w/w, about 29.7% w/w, about 29.8% w/w, about 29.9% w/w, about 30.0% w/w, 30.1% w/w, about 30.2% w/w, about 30.3% w/w, about 30.4% w/w, about 30.5% w/w, about 30.6% w/w, about 30.7% w/w, about 30.8% w/w, about 30.9% w/w, about 31.0% w/w, 31.1% w/w, about 31.2% w/w, about 31.3% w/w, about 31.4% w/w, about 31.5% w/w, about 31.6% w/w, about 31.7% w/w, about 31.8% w/w, about 31.9% w/w, about 32.0% w/w, 32.1% w/w, about 32.2% w/w, about 32.3% w/w, about 32.4% w/w, about 32.5% w/w, about 32.6% w/w, about 32.7% w/w, about 32.8% w/w, about 32.9% w/w, about 33.0% w/w, 33.1% w/w, about 33.2% w/w, about 33.3% w/w, about 33.4% w/w, about 33.5% w/w, about 33.6% w/w, about 33.7% w/w, about 33.8% w/w, about 33.9% w/w, about 34.0% w/w, 34.1% w/w, about 34.2% w/w, about 34.3% w/w, about 34.4% w/w, about 34.5% w/w, about 34.6% w/w, about 34.7% w/w, about 34.8% w/w, about 34.9% w/w, about 35.0% w/w, 35.1% w/w, about 35.2% w/w, about 35.3% w/w, about 35.4% w/w, about 35.5% w/w, about 35.6% w/w, about 35.7% w/w, about 35.8% w/w, about 35.9% w/w, about 36.0% w/w, 36.1% w/w, about 36.2% w/w, about 36.3% w/w, about 36.4% w/w, about 36.5% w/w, about 36.6% w/w, about 36.7% w/w, about 36.8% w/w, about 36.9% w/w, about 37.0% w/w, 37.1% w/w, about 37.2% w/w, about 37.3% w/w, about 37.4% w/w, about 37.5% w/w, about 37.6% w/w, about 37.7% w/w, about 37.8% w/w, about 37.9% w/w, about 38.0% w/w, 38.1% w/w, about 38.2% w/w, about 38.3% w/w, about 38.4% w/w, about 38.5% w/w, about 38.6% w/w, about 38.7% w/w, about 38.8% w/w, about 38.9% w/w, about 39.0% w/w, 39.1% w/w, about 39.2% w/w, about 39.3% w/w, about 39.4% w/w, about 39.5% w/w, about 39.6% w/w, about 39.7% w/w, about 39.8% w/w, about 39.9% w/w, about 40.0% w/w, 40.1% w/w, about 40.2% w/w, about 40.3% w/w, about 40.4% w/w, about 40.5% w/w, about 40.6% w/w, about 40.7% w/w, about 40.8% w/w, about 40.9% w/w, about 41.0% w/w, 41.1% w/w, about 41.2% w/w, about 41.3% w/w, about 41.4% w/w, about 41.5% w/w, about 41.6% w/w, about 41.7% w/w, about 41.8% w/w, about 41.9% w/w, about 42.0% w/w, 42.1% w/w, about 42.2% w/w, about 42.3% w/w, about 42.4% w/w, about 42.5% w/w, about 42.6% w/w, about 42.7% w/w, about 42.8% w/w, about 42.9% w/w, about 43.0% w/w, 43.1% w/w, about 43.2% w/w, about 43.3% w/w, about 43.4% w/w, about 43.5% w/w, about 43.6% w/w, about 43.7% w/w, about 43.8% w/w, about 43.9% w/w, about 44.0% w/w, 44.1% w/w, about 44.2% w/w, about 44.3% w/w, about 44.4% w/w, about 44.5% w/w, about 44.6% w/w, about 44.7% w/w, about 44.8% w/w, about 44.9% w/w, about 45.0% w/w, 41.1% w/w, about 41.2% w/w, about 41.3% w/w, about 41.4% w/w, about 41.5% w/w, about 41.6% w/w, about 41.7% w/w, about 41.8% w/w, about 41.9% w/w, about 42.0% w/w, 42.1% w/w, about 42.2% w/w, about 42.3% w/w, about 42.4% w/w, about 42.5% w/w, about 42.6% w/w, about 42.7% w/w, about 42.8% w/w, about 42.9% w/w, about 43.0% w/w, 43.1% w/w, about 43.2% w/w, about 43.3% w/w, about 43.4% w/w, about 43.5% w/w, about 43.6% w/w, about 43.7% w/w, about 43.8% w/w, about 43.9% w/w, about 44.0% w/w, 44.1% w/w, about 44.2% w/w, about 44.3% w/w, about 44.4% w/w, about 44.5% w/w, about 44.6% w/w, about 44.7% w/w, about 44.8% w/w, about 44.9% w/w, about 45.0% w/w, 45.1% w/w, about 45.2% w/w, about 45.3% w/w, about 45.4% w/w, about 45.5% w/w, about 45.6% w/w, about 45.7% w/w, about 45.8% w/w, about 45.9% w/w, about 46.0% w/w, 46.1% w/w, about 46.2% w/w, about 46.3% w/w, about 46.4% w/w, about 46.5% w/w, about 46.6% w/w, about 46.7% w/w, about 46.8% w/w, about 46.9% w/w, about 47.0% w/w, 47.1% w/w, about 47.2% w/w, about 47.3% w/w, about 47.4% w/w, about 47.5% w/w, about 47.6% w/w, about 47.7% w/w, about 47.8% w/w, about 47.9% w/w, about 48.0% w/w, 48.1% w/w, about 48.2% w/w, about 48.3% w/w, about 48.4% w/w, about 48.5% w/w, about 48.6% w/w, about 48.7% w/w, about 48.8% w/w, about 48.9% w/w, about 49.0% w/w, 49.1% w/w, about 49.2% w/w, about 49.3% w/w, about 49.4% w/w, about 49.5% w/w, about 49.6% w/w, about 49.7% w/w, about 49.8% w/w, about 49.9% w/w, about 50.0% w/w, 50.1% w/w, about 50.2% w/w, about 50.3% w/w, about 50.4% w/w, about 50.5% w/w, about 50.6% w/w, about 50.7% w/w, about 50.8% w/w, about 50.9% w/w, about 51.0% w/w, 51.1% w/w, about 51.2% w/w, about 51.3% w/w, about 51.4% w/w, about 51.5% w/w, about 51.6% w/w, about 51.7% w/w, about 51.8% w/w, about 51.9% w/w, about 52.0% w/w, 52.1% w/w, about 52.2% w/w, about 52.3% w/w, about 52.4% w/w, about 52.5% w/w, about 52.6% w/w, about 52.7% w/w, about 52.8% w/w, about 52.9% w/w, about 53.0% w/w, 53.1% w/w, about 53.2% w/w, about 53.3% w/w, about 53.4% w/w, about 53.5% w/w, about 53.6% w/w, about 53.7% w/w, about 53.8% w/w, about 53.9% w/w, about 54.0% w/w, 54.1% w/w, about 54.2% w/w, about 54.3% w/w, about 54.4% w/w, about 54.5% w/w, about 54.6% w/w, about 54.7% w/w, about 54.8% w/w, about 54.9% w/w, about 55.0% w/w, 55.1% w/w, about 55.2% w/w, about 55.3% w/w, about 55.4% w/w, about 55.5% w/w, about 55.6% w/w, about 55.7% w/w, about 55.8% w/w, about 55.9% w/w, about 56.0% w/w, 56.1% w/w, about 56.2% w/w, about 56.3% w/w, about 56.4% w/w, about 56.5% w/w, about 56.6% w/w, about 56.7% w/w, about 56.8% w/w, about 56.9% w/w, about 57.0% w/w, 57.1% w/w, about 57.2% w/w, about 57.3% w/w, about 57.4% w/w, about 57.5% w/w, about 57.6% w/w, about 57.7% w/w, about 57.8% w/w, about 57.9% w/w, about 58.0% w/w, 58.1% w/w, about 58.2% w/w, about 58.3% w/w, about 58.4% w/w, about 58.5% w/w, about 58.6% w/w, about 58.7% w/w, about 58.8% w/w, about 58.9% w/w, about 59.0% w/w, 59.1% w/w, about 59.2% w/w, about 59.3% w/w, about 59.4% w/w, about 59.5% w/w, about 59.6% w/w, about 59.7% w/w, about 59.8% w/w, about 59.9% w/w, about 60.0% w/w, 60.1% w/w, about 60.2% w/w, about 60.3% w/w, about 60.4% w/w, about 60.5% w/w, about 60.6% w/w, about 60.7% w/w, about 60.8% w/w, about 60.9% w/w, about 61.0% w/w, 61.1% w/w, about 61.2% w/w, about 61.3% w/w, about 61.4% w/w, about 61.5% w/w, about 61.6% w/w, about 61.7% w/w, about 61.8% w/w, about 61.9% w/w, about 62.0% w/w, 62.1% w/w, about 62.2% w/w, about 62.3% w/w, about 62.4% w/w, about 62.5% w/w, about 62.6% w/w, about 62.7% w/w, about 62.8% w/w, about 62.9% w/w, about 63.0% w/w, 63.1% w/w, about 63.2% w/w, about 63.3% w/w, about 63.4% w/w, about 63.5% w/w, about 63.6% w/w, about 63.7% w/w, about 63.8% w/w, about 63.9% w/w, about 64.0% w/w, 64.1% w/w, about 64.2% w/w, about 64.3% w/w, about 64.4% w/w, about 64.5% w/w, about 64.6% w/w, about 64.7% w/w, about 64.8% w/w, about 64.9% w/w, about 65.0% w/w, 65.1% w/w, about 65.2% w/w, about 65.3% w/w, about 65.4% w/w, about 65.5% w/w, about 65.6% w/w, about 65.7% w/w, about 65.8% w/w, about 65.9% w/w, about 66.0% w/w, 66.1% w/w, about 66.2% w/w, about 66.3% w/w, about 66.4% w/w, about 66.5% w/w, about 66.6% w/w, about 66.7% w/w, about 66.8% w/w, about 66.9% w/w, about 67.0% w/w, 67.1% w/w, about 67.2% w/w, about 67.3% w/w, about 67.4% w/w, about 67.5% w/w, about 67.6% w/w, about 67.7% w/w, about 67.8% w/w, about 67.9% w/w, about 68.0% w/w, 68.1% w/w, about 68.2% w/w, about 68.3% w/w, about 68.4% w/w, about 68.5% w/w, about 68.6% w/w, about 68.7% w/w, about 68.8% w/w, about 68.9% w/w, about 69.0% w/w, 69.1% w/w, about 69.2% w/w, about 69.3% w/w, about 69.4% w/w, about 69.5% w/w, about 69.6% w/w, about 69.7% w/w, about 69.8% w/w, about 69.9% w/w, about 70.0% w/w, 70.1% w/w, about 70.2% w/w, about 70.3% w/w, about 70.4% w/w, about 70.5% w/w, about 70.6% w/w, about 70.7% w/w, about 70.8% w/w, about 70.9% w/w, about 71.0% w/w, 71.1% w/w, about 71.2% w/w, about 71.3% w/w, about 71.4% w/w, about 71.5% w/w, about 71.6% w/w, about 71.7% w/w, about 71.8% w/w, about 71.9% w/w, about 72.0% w/w, 72.1% w/w, about 72.2% w/w, about 72.3% w/w, about 72.4% w/w, about 72.5% w/w, about 72.6% w/w, about 72.7% w/w, about 72.8% w/w, about 72.9% w/w, about 73.0% w/w, 73.1% w/w, about 73.2% w/w, about 73.3% w/w, about 73.4% w/w, about 73.5% w/w, about 73.6% w/w, about 73.7% w/w, about 73.8% w/w, about 73.9% w/w, about 74.0% w/w, 74.1% w/w, about 74.2% w/w, about 74.3% w/w, about 74.4% w/w, about 74.5% w/w, about 74.6% w/w, about 74.7% w/w, about 74.8% w/w, and about 74.9% w/w.

In certain aspects, the active agent (or payload) may comprise a carbonate, e.g., a pH increasing composition or an alkalinity increasing composition, where the carbonate serves a dual function—namely, increase the pH of the recreational water and facilitate disintegration of the composition. Accordingly, another aspect relates to a composition comprising from about 20% w/w to about 85% w/w of a carbonate and all values in between, as indicated above.

The first embodiment may comprise from about 1% w/w to about 30% w/w w/w of a skin oil, a fragrance, a surfactant, an adjuvant, or a combination thereof. For instance, the rapidly disintegrating buoyant composition may comprise one or more of a skin oil, a fragrance, a surfactant, and an adjuvant.

In one aspect, the rapidly disintegrating buoyant composition comprises from about 1% w/w to about 30% w/w of a skin oil, a fragrance, a surfactant, an adjuvant, or a combination thereof.

In one aspect, the rapidly disintegrating buoyant composition comprises from about 1% w/w to about 8% w/w of a skin oil, a fragrance, a surfactant, or a combination thereof.

Examples of suitable skin oils include, but are not limited to, an avocado butter, an avocado oil, an almond butter, an almond oil, an olive butter, an olive oil, a shea butter, a shea oil, a cocoa butter, a cocoa oil, a coconut butter, a coconut oil, and the like.

Skin oils may be present in an amount that ranges from about 1% w/w to about 5% w/w, and all values in between, including about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, and about 19% w/w.

One will appreciate that shea butter extract is a complex fat that may comprise the following fatty acids: oleic acid (40-60%), stearic acid (20-50%), linoleic acid (3-11%), palmitic acid (2-9%), linolenic acid (<1%) and arachidic acid (<1%).

One also will appreciate that cocoa butter may comprise oleic acid (34.5%), stearic acid (34.5%), palmitic acid (26.0%), linoleic acid (3.2%), arachidic acid (1.0%), other fatty acids (0.5%), palmitoleic acid (0.3%).

Examples of suitable fragrances include, but are not limited to, Starburst (e.g., 5991044 commercially available from International Flavors & Fragrances, Inc.), Island Cooler (e.g., 455778 commercially available from Symrise), Tropical Fresh Pool (e.g., E20055772 from Mane), and the like.

A fragrance may be present in an amount that ranges from about 1% w/w to about 5% w/w, and all values in between, including about 1% w/w, about 2% w/w, about 3% w/w, and about 4% w/w.

Examples of suitable surfactants may include, for example, an anionic surfactant, a cationic surfactant, a non-nonionic surfactant, a zwitterionic surfactant, an N-oxide surfactant, or a combination thereof.

A surfactant may be present in an amount that ranges from about 1% w/w to about 5% w/w, and all values in between, including about 1% w/w, about 2% w/w, about 3% w/w, and about 4% w/w.

An adjuvant may be present in an amount that ranges from about 0% w/w to about 30% w/w.

Examples of suitable adjuvants include, but are not limited to, sodium gluconate, edetic acid (“EDTA”), an EDTA salt (e.g., sodium edetate, disodium edetate, trisodium edetate, edetate calcium disodium, and the like), N-(1-Carboxyethyl)-iminodiacetic acid (MGDA), or a MGDA salt (e.g., trisodium dicarboxymethyl alaninate), a conjugate base (e.g., a salt) of an organic acid disclosed herein (e.g., aconitate, adipate, ascorbate, benzoate, citrate (e.g., sodium citrate), gluconate, glutarate, glycerate, lactate, malate, malonate, succinate, tartrate, or a combination thereof) and the like.

In one aspect, a clarifier composition comprises an aluminum sulfate (about 19.5% w/w), citric acid (from about 23% w/w to about 47% w/w), sodium bicarbonate (from about 32% w/w to about 56% w/w), a fragrance (about 0.9% w/w), shea butter (about 0.5% w/w), and a surfactant (about 0.3% w/w), wherein the equivalent ratio of citric acid to carbonate ranges from about 0.3 to about 1.1.

In one aspect, a clarifier composition comprises an aluminum sulfate (about 19.5% w/w), citric acid (about 12.6% w/w), sodium bicarbonate (about 66.1% w/w), a fragrance (about 1% w/w), shea butter (about 0.5% w/w), and a surfactant (about 0.3% w/w).

In a further aspect, a clarifier composition comprises an aluminum sulfate (about 19.5% w/w), citric acid (about 12.6% w/w), sodium bicarbonate (about 66.1% w/w), a fragrance (about 1% w/w), shea butter (about 0.5% w/w), and tomadol 1-9 (0.3% w/w).

In another aspect, a clarifier composition comprises an aluminum sulfate (about 19.5% w/w), citric acid (about 59.3% w/w), sodium bicarbonate (about 18.1% w/w), a fragrance (about 1% w/w), shea butter (about 0.5% w/w), and a surfactant (about 0.3% w/w).

In yet another aspect, a clarifier composition comprises an aluminum sulfate (about 19.5% w/w), citric acid (about 59.3% w/w), sodium bicarbonate (about 18.1% w/w), a fragrance (about 1% w/w), shea butter (about 0.5% w/w), and tomadol 1-9 (0.3% w/w).

In one aspect, a clarifier composition comprises an aluminum sulfate (about 19.5% w/w), citric acid (about 22.6% w/w), sodium carbonate (about 56.2% w/w), a fragrance (about 0.9% w/w), shea butter (about 0.5% w/w), and a surfactant (about 0.3% w/w), wherein the equivalent ratio of citric acid to carbonate is about 0.3.

One will appreciate that certain compositions may include a limited number of ingredients, and thus, one may appreciate that the composition may consist the stated ingredients. As an example, one aspect relates to a clarifier composition consists of an aluminum sulfate (about 19.5% w/w), citric acid (about 22.6% w/w), sodium carbonate (about 56.2% w/w), a fragrance (about 0.9% w/w), shea butter (about 0.5% w/w), and a surfactant (about 0.3% w/w), wherein the equivalent ratio of citric acid to carbonate is about 0.3.

In another aspect, a clarifier composition comprises an aluminum sulfate (about 19.5% w/w), citric acid (about 34.8% w/w), sodium carbonate (about 43.9% w/w), a fragrance (about 0.9% w/w), shea butter (about 0.5% w/w), and a surfactant (about 0.3% w/w), wherein the equivalent ratio of citric acid to carbonate is about 0.6.

In another aspect, a clarifier composition comprises an aluminum sulfate (about 19.5% w/w), citric acid (about 46.7% w/w), sodium carbonate (about 32.1% w/w), a fragrance (about 0.9% w/w), shea butter (about 0.5% w/w), and a surfactant (about 0.3% w/w), wherein the equivalent ratio of citric acid to carbonate is about 1.1.

One aspect relates to an algicide composition, which may comprise a copper sulfate, a copper citrate, an ADBAC, as stated herein, where the amount of algicide depends on the desired concentration in the body of recreational water. One may appreciate that a suitable range for a copper-based algicide in a body of recreational water may range of from about 0.1 ppm to 0.7 ppm. Further, one may appreciate that a suitable range for an ADBAC-based algicide in a body of recreational water may range of from about 0.25 ppm to about 5 ppm (or more). For instance, an algicide composition intended to treat about 10,000 gallons of recreational water comprises about 37.8 g of CuSO₄·5H₂O, which would result in a copper (Cu²⁺) concentration of about 0.25 ppm, while an algicide composition intended to treat about 5,000 gallons of recreational water comprises about 18.9 g of CuSO₄·5H₂O.

In view of the foregoing, one aspect relates to an algicide composition comprises copper sulfate (23.7% w/w), citric acid (about 33.4% w/w), sodium bicarbonate (about 41.7% w/w), a fragrance (about 0.8% w/w), and shea butter (about 0.4% w/w).

In one aspect, a pH reducing composition comprises citric acid (about 73.9% w/w), sodium bicarbonate (about 24.4% w/w), a fragrance (about 0.9% w/w), shea butter (about 0.5% w/w), and a surfactant (about 0.3% w/w).

In another aspect, a pH reducing composition consists of citric acid (about 73.9% w/w), sodium bicarbonate (about 24.4% w/w), a fragrance (about 0.9% w/w), shea butter (about 0.5% w/w), and a surfactant (about 0.3% w/w).

In one aspect, an alkalinity increasing composition comprises citric acid (about 15.7% w/w), sodium bicarbonate (about 82.6% w/w), a fragrance (about 0.9% w/w), shea butter (about 0.5% w/w), and a surfactant (about 0.3% w/w).

In another aspect, an alkalinity increasing composition consists of citric acid (about 15.7% w/w), sodium bicarbonate (about 82.6% w/w), a fragrance (about 0.9% w/w), shea butter (about 0.5% w/w), and a surfactant (about 0.3% w/w).

In one aspect, a pH increasing composition comprises citric acid (22.8% w/w), sodium carbonate (75.5% w/w), a fragrance (0.9% w/w), shea butter (0.5% w/w), and a surfactant (0.3% w/w).

In another aspect, a pH increasing composition consists of citric acid (22.8% w/w), sodium carbonate (75.5% w/w), a fragrance (0.9% w/w), shea butter (0.5% w/w), and a surfactant (0.3% w/w).

The rapidly disintegrating buoyant composition may be formed in a suitable shape, including for example, a spherical tablet, a substantially spherical tablet, an elongated tablet (e.g., a puck), a substantially cylindrical tablet, and the like. In one aspect, a compacted tablet (about 3 inches) weighs about 8 ounces (about 227 g). In another aspect, a tablet having a thickness of about 1.5 inches and a about 3-inch diameter provides an approximate volume (assuming a right-circular cylinder) of about 10 cubic inches (about 174 cm³ volume). In one aspect, the amount of tablet material ranges from about 150 g to about 175 g, and all values in between, including, for example about 155 g. about 160 g, about 165 g, and about 170 g. This shape is robust for manufacture and transport, easy to handle, and has been a long-term industry standard such that it is compatible with the majority of pool operation and maintenance equipment. It is also relatively efficient from a surface to volume ratio, and the large flat faces allow easy stacking which can be tailored to meet the needs of a specific pool installation. Therefore, there is little advantage to be gained from modifying the tablet shape.

One aspect of the first embodiment relates to a shaped composition obtained by a process that comprises applying a pressure of about 60 psi to about 500 psi to obtain a shaped rapidly disintegrating buoyant composition. The pressure to be applied to form a shaped composition may range from about 60 psi to about 500 psi, and all values in between, including for example, about 70 psi, about 80 psi, about 90 psi, about 100 psi, and about 110 psi, about 120 psi, about 140 psi, about 160 psi, about 180 psi, about 200 psi, about 220 psi, about 240 psi, about 260 psi, about 280 psi, about 300 psi, about 320 psi, about 340 psi, about 360 psi, about 380 psi, about 400 psi, about 420 psi, about 440 psi, about 460 psi, and about 480 psi. For instance, the applied pressure to form a shaped composition may be about 90 psi. Further, the applied pressure to form a shaped composition may be about 110 psi.

A second embodiment relates to a process for preparing a formed composition, comprising: a) an active agent, b) at least about 15% w/w of an organic acid, c) at least about 10% w/w of a carbonate, and d) about 1% w/w to about 30% w/w of a skin oil, a fragrance, a surfactant, an adjuvant, or a combination thereof, which comprises: (i) mixing the stated ingredients a)-d) to obtain a mixed composition: (ii) adding a solution to the mixed composition to obtain a moistened solid mixture: (iii) blending the moistened solid mixture to obtain a mixed solid mixture: (iv) adding a portion of the mixed solid mixture to a mold to obtain a molded, mixed solid mixture: (v) applying a pressure of about 60 psi to about 500 psi to the molded, mixed solid mixture to obtain a shaped composition; and (iv) drying the shaped composition to obtain a shaped rapidly dissolving buoyant composition.

An alternative second embodiment relates to a process for preparing a formed composition, comprising: a) an active agent, b) at least about 15% w/w of an organic acid, c) at least about 10% w/w of a carbonate, and d) about 1% w/w to about 30% w/w of a skin oil, a fragrance, a surfactant, an adjuvant, or a combination thereof, which comprises: (i) mixing the stated ingredients a)-d) to obtain a mixed composition: (ii) adding a solution to the mixed composition to obtain a moistened solid mixture: (iii) blending the moistened solid mixture to obtain a mixed solid mixture: (iv) drying the mixed solid mixture to obtain a dried mixture: (v) adding a portion of the dried mixture to a mold; and (vi) applying a pressure of about 60 psi to about 500 psi to obtain the shaped rapidly disintegrating buoyant composition.

One will appreciate that the amounts of ingredients identified for any of the aspects of the first embodiment may be utilized in the formation of a shaped composition of the second embodiment.

Different blender types providing adequate preparation mixing include, but are not limited to, batch or continuous blenders such as twin-shell V-blenders, twin-shelled cross-flow blenders, screw blenders, and screw conveyer flow blenders. In one aspect, the mixing may be obtained using a twin-shell V-blender, a twin-shelled cross-flow blender, a screw blender, a screw conveyer flow blender, and the like.

In one aspect, the solution may be added to the mixed composition by any suitable method that provides a uniform application of the solution, e.g., spraying, squirting, misting, and the like.

The amount of solution depends on the amount of mixed composition. In one aspect, the amount of solution ranges from about 1% w/w to about 5% w/w based on the weight of the batch composition, and all values in between, e.g., about 2% w/w; about 3% w/w; and about 4% w/w. For instance, an amount of solution that ranges from about 4 g to about 20 g may be applied to a batch size of about 450 g.

In one aspect, the solution of the second embodiment comprises water and a miscible organic solvent. A miscible organic solvent comprises methanol, ethanol, isopropyl alcohol, acetone, propylene glycol, and the like, or a combination thereof. The amount of water in the solution ranges from about 10% v/v to about 40% v/v, including all values in between, for example, about 15% v/v, about 20% v/v, about 25% v/v, about 30% v/v, and about 35% v/v, and the amount of the miscible organic solvent being the balance of the solution. The amount of water in the solution may depend on the amount of organic acid and carbonate at least because introduction of water may result in an initiation of effervescence caused by the reaction of the organic acid with the carbonate. Observations showed that an increased water content in the solution helps create a better (less friable) tablet, but too much water initiated the effervescence during manufacture, such that, when applied, the tablet fizzed very little, if at all.

In one aspect, the drying may be obtained by any suitable method, including, for example, oven drying, air drying, ambient drying, and the like. One may appreciate that the drying may be accomplished when a loss on drying (LOD) reaches a constant value. In one aspect, the water content of the dried, shaped composition ranges from about 0.1% to about 2% w/w, and all values in between, including, for example, about 0.2% w/w, about 0.3% w/w, about 0.4% w/w, about 0.5% w/w, about 0.6% w/w; about 0.7% w/w, about 0.8% w/w, about 0.9% w/w; about 1.0% w/w; about 1.1% w/w, about 1.2% w/w, about 1.3% w/w, about 1.4% w/w, about 1.5% w/w; about 1.6% w/w, about 1.7% w/w, about 1.8% w/w, and about 1.9% w/w.

In one aspect, the mold of the second embodiment has a shape capable of preparing a suitably formed composition, including for example, a spherical tablet, a substantially spherical tablet, an elongated tablet (e.g., a puck), a substantially cylindrical tablet, and the like.

In one aspect, the pressure application ranges from about 60 psi to about 500 psi, and all values in between, including, for example, about 70 psi, about 80 psi, about 90 psi, about 100 psi, and about 110 psi, about 120 psi, about 140 psi, about 160 psi, about 180 psi, about 200 psi, about 220 psi, about 240 psi, about 260 psi, about 280 psi, about 300 psi, about 320 psi, about 340 psi, about 360 psi, about 380 psi, about 400 psi, about 420 psi, about 440 psi, about 460 psi, and about 480 psi. For instance, the applied pressure to form a shaped composition may be about 90 psi. Further, the applied pressure to form a shaped composition may be about 110 psi.

The pressure application may be achieved using a suitable press, e.g., a pneumatic press, or the like. Not to be limited by theory, it is believed that the formed composition obtained by applying a pressure of about 60 psi to about 500 psi may improve the porosity of the composition, thus, allowing an ingress of water to initiate effervescence and promote buoyancy of the formed composition. Observations revealed that for certain compositions a higher pressure (e.g., greater than about 120 psi to about 500 psi) in molding the formed composition decreased the tendency to float. However, a lower pressure (e.g., less than about 60 psi) created a formed composition that fell apart as it dried or as it was handled.

A third embodiment relates to a formed composition obtained by a process comprising: a) an active agent, b) at least about 15% w/w of an organic acid, c) at least about 10% w/w of a carbonate, and d) about 1% w/w to about 30% w/w of a skin oil, a fragrance, a surfactant, an adjuvant, or a combination thereof, which comprises: (i) mixing the stated ingredients a)-d) to obtain a mixed composition, (ii) spraying the mixed composition with a solution to obtain a moistened solid mixture, (iii) blending the moistened solid mixture to obtain a mixed solid mixture, (iv) adding a portion of the mixed solid mixture to a mold to obtain a molded, mixed solid mixture: (v) applying a pressure of about 60 psi to about 500 psi to the molded, mixed solid mixture to obtain a shaped composition; and (iv) drying the shaped composition to obtain a shaped rapidly dissolving buoyant composition.

An alternative third embodiment relates to a formed composition obtained by a process comprising: a) an active agent, b) at least about 15% w/w of an organic acid, c) at least about 10% w/w of a carbonate, and d) about 1% w/w to about 30% w/w of a skin oil, a fragrance, a surfactant, an adjuvant, or a combination thereof, which comprises: (i) mixing the stated ingredients a)-d) to obtain a mixed composition, (ii) spraying the mixed composition with a solution to obtain a moistened solid mixture, (iii) blending the moistened solid mixture to obtain a mixed solid mixture, (iv) drying the mixed solid mixture to obtained a dried mixture: (v) adding a portion of the dried mixture to a mold; and (vi) applying a pressure of about 60 psi to about 500 psi to obtain the shaped rapidly disintegrating buoyant composition.

One will appreciate that the amounts of ingredients identified for any of the aspects of the first embodiment may be utilized in the formation of a shaped composition of the third embodiment. One also will appreciate that any stated process parameters may be applicable to the formed composition of the third embodiment.

A fourth embodiment relates to a method for treating a recirculating water system, which comprises: adding any one of the formed compositions described herein to the water of the recirculating water system.

One will appreciate that any one of the compositions of the first and third embodiments may be utilized in the method of the fourth embodiment.

### EXAMPLES

The following information illustrates aspects of the rapidly disintegrating buoyant compositions disclosed herein and should not be considered to be limiting on the subject matter claimed herein.

At the outset, it was believed that composition density may be modified using a hydrophobic material providing a density close to the density of water (e.g., 1 g/cm³). To that end, compositions having a higher amount of hydrophobic materials greatly reduced the effervescence and the buoyancy of the tablet. Indeed, experiments show that density considerations alone may not result in a rapidly disintegrating buoyant composition.

Take for example a substantially spherical table having a diameter of about 2.5-inches, which was manufactured using a hydraulic press and a pneumatic press. The pneumatic press was set to 90 pounds per square inch (“psi”) pressure. The hydraulic press was set to 21,000 lbs pressure (which for a 2.5″ tablet mold gives approximately 4300 psi pressure). The rough volume of a 2.5″ sphere is approximately 134 cubic centimeters. One of the heaviest floating tablets had a mass of about 118.92 g (density of about 0.89 g/cm³). The lightest sinking tab was 161.5 g (density of about 1.21 g/cm³). Interestingly, density is not the only variable that causes the tablet to float. For instance, a detailed design of experiment (“DoE”) showed a tablet having a high proportion of cocoa butter with a density of about 0.90 g/cm³-based on density alone, the tablet should have floated. Instead, the cocoa butter containing tablet sank. Thus, the DoE study showed that bulk composition density alone does not dictate whether a tablet sinks or floats. Instead, factors that influence buoyancy include density, porosity, effervescence, and friability.

Substantially spherical tablets were manufactured and comprised of 141.8 g copper sulfate (viz., CuSO₄·5H₂O), 5 mL of fragrance, and 2.5 mL of shea butter, along with the ingredients (viz., citric acid, sodium carbonate (Na₂CO₃), sodium gluconate (SG), disodium citrate (“DC”)) in the stated amounts.

| | Citric Acid | Na₂CO₃ | | DSC | Observation post |
|---|---|---|---|---|---|
| Tablet | (g) | (g) | SG (g) | (g) | stability^{†} |
| A | 100 | 150 | 0 | 0 | X |
| B | 100 | 150 | 0 | 50 | AC |
| C | 100 | 150 | 125 | 0 | AC |
| D | 100 | 150 | 125 | 50 | AC |
| E | 100 | 250 | 0 | 0 | FDNF |
| F | 100 | 250 | 0 | 50 | FDNF |
| G | 100 | 250 | 125 | 0 | AC |
| H | 100 | 250 | 125 | 50 | AC/X |
| I | 200 | 150 | 0 | 0 | DNFDNF |
| J | 200 | 150 | 0 | 50 | DNFDNF |
| K | 200 | 150 | 125 | 0 | AC |
| L | 200 | 150 | 125 | 50 | AC |
| M | 200 | 250 | 0 | 0 | RD |
| N | 200 | 250 | 0 | 50 | RD |
| O | 200 | 250 | 125 | 0 | AC |
| P | 200 | 250 | 125 | 50 | AC |

^{†}Post-stability observations conducted after tablet storage for one-month at about 33° C. and about 65% relative humidity.
Key: X (tablet fell apart), AC (appearance changed), FDNF, (fizzed, did not float), DNFDNF (did not fizz, did not float), BF (barely fizzed), RD (rapid dissolution).

Tablets were manufactured by mixing the solid ingredients (e.g., copper sulfate, citric acid, Na₂CO₃, and optionally sodium gluconate (“SG”) and disodium citrate (“DC”)) to form a solid mixture. Fragrance and shea butter were blended into the solid mixture. The solid mixture was sprayed with a mist a solution of water (30% v/v) dissolved in isopropyl alcohol (70% v/v). The amount of solution ranged from about 2% w/w to about 5% w/w where the percentage of solution was based on the amount of solid mixture. The moistened solid mixture was blended thoroughly to disperse the moisture throughout the moistened solid mixture to obtain the moistened solid mixture having the consistency similar to wet sand. The mixture was then added to a mold and pressed using a pneumatic press with a compaction pressure of about 90 psi. Manufactured tablets may be dried in an oven or at room temperature.

Formed tablets had a weight of about 118±2 g. Formed tablets were subjected to stability testing at about 33° C. and 65% relative humidity (“RH”). A tablet composition was considered to be suitable for commercial use when there was no change in appearance and the tablet floated and fizzed. Compositions disclosed and claimed herein exhibit buoyancy with rapid disintegration when stored at 33° C./65% RH for one month.

### DISCLOSED ASPECTS

Aspect 1. A rapidly disintegrating buoyant composition, comprising: a) an active agent, b) at least about 15% w/w of an organic acid, c) at least about 10% w/w of a carbonate, and d) about 1% w/w to about 30% w/w of a skin oil, a fragrance, a surfactant, an adjuvant, or a combination thereof.

Aspect 2. The rapidly disintegrating buoyant composition of Aspect 1, wherein the rapidly disintegrating buoyant composition has an equivalent ratio of the organic acid to the carbonate ranges from about 0.3 to about 1.1.

Aspect 3. The rapidly disintegrating buoyant composition of any one of Aspects 1-2, wherein the rapidly disintegrating buoyant composition has an equivalent ratio of the organic acid to the carbonate of about 0.6 to about 1.1.

Aspect 4. The rapidly disintegrating buoyant composition of any one of Aspects 1-3, wherein active agent comprises a clarifier, an algicide, a phosphate removal composition, a surfactant, an enzyme, a purifying agent, a scale reducing agent, a flocculant, a chelating agent, alkalinity stabilizing agent, non-chlorine oxidant, a fragrance, a dye, a water stabilizer/conditioner, a calcium hardness adjusting agent, a buffering agent, a CYA removal agent, a chlorine and/or chloramine reducing agent, or a combination thereof.

Aspect 5. The rapidly disintegrating buoyant composition of any one of Aspects 1-4, wherein the organic acid comprises aconitic acid, adipic acid, ascorbic acid, benzoic acid, citric acid, gluconic acid, glutaric acid, glyceric acid, lactic acid, malic acid, malonic acid, succinic acid, tartaric acid, or a combination thereof.

Aspect 6. The rapidly disintegrating buoyant composition of any one of Aspects 1-5, wherein the organic acid is present in an amount of from about 20% w/w to about 75% w/w.

Aspect 7. The rapidly disintegrating buoyant composition of any one of Aspects 1-6, wherein the carbonate comprises a bicarbonate, a carbonate, a sesquicarbonate, or a combination thereof.

Aspect 8. The rapidly disintegrating buoyant composition of any one of Aspects 1-7, wherein the carbonate is present in an amount of from about 10% w/w to about 75% w/w.

Aspect 9. The rapidly disintegrating buoyant composition of any one of Aspects 1-8, wherein the skin oil comprises an avocado butter, an avocado oil, an almond butter, an almond oil, an olive butter, an olive oil, a shea butter, a shea oil, a cocoa butter, a cocoa oil, a coconut butter, a coconut oil, or a combination thereof.

Aspect 10. The rapidly disintegrating buoyant composition of any one of Aspects 1-9, wherein the skin oil ranges from about 1% w/w to about 5% w/w.

Aspect 11. The rapidly disintegrating buoyant composition of any one of Aspects 1-10, wherein the fragrance ranges from about 1% w/w to about 5% w/w.

Aspect 12a. The rapidly disintegrating buoyant composition of any one of Aspects 1-11, wherein the surfactant comprises a non-ionic surfactant, a cationic surfactant, an anionic surfactant, a zwitterionic surfactant, an N-oxide surfactant, or a combination thereof.

Aspect 12b. The rapidly disintegrating buoyant composition of any one of Aspects 1-11, wherein the surfactant comprises a polyethylene glycol undecyl ether, an α-hydro-ω-hydroxypoly(oxyethylene)poly(oxypropylene)poly(oxyethylene) block copolymer, an α-(nonylphenyl)-ω-hydroxy-poly(oxy-1,2-ethanediyl), a polyoxyethylene sorbitan fatty acid ester a polyoxyalkylene glycol oleic acid condensate, or a combination thereof.

Aspect 13. The rapidly disintegrating buoyant composition of any one of Aspects 1-12, wherein the surfactant ranges from about 1% w/w to about 5% w/w.

Aspect 14. The rapidly disintegrating buoyant composition of any one of Aspects 1-13, wherein the adjuvant comprises sodium gluconate, edetic acid, salt of edetic acid, a N-(1-Carboxyethyl)-iminodiacetic acid, a salt of N-(1-Carboxyethyl)-iminodiacetic acid, a salt of aconitic acid, a salt of adipic acid, a salt of ascorbic acid, a salt of benzoic acid, a salt of citric acid, a salt of gluconic acid, a salt of glutaric acid, a salt of glyceric acid, a salt of lactic acid, a salt of malic acid, a salt of malonic acid, a salt of succinic acid, a salt of tartaric acid, or a combination thereof.

Aspect 15. The rapidly disintegrating buoyant composition of any one of Aspects 1-14, the adjuvant ranges from about 0% w/w to about 30% w/w.

Aspect 16. The rapidly disintegrating buoyant composition of Aspect 1 comprising an aluminum sulfate (about 19.5% w/w), citric acid (from about 23% w/w to about 47% w/w), sodium bicarbonate (from about 32% w/w to about 56% w/w), a fragrance (about 0.9% w/w), shea butter (about 0.5% w/w), and a surfactant (about 0.3% w/w), wherein the equivalent ratio of citric acid to carbonate ranges from about 0.3 to about 1.1.

Aspect 17. The rapidly disintegrating buoyant composition of Aspect 1 comprising an aluminum sulfate (about 19.5% w/w), citric acid (about 12.6% w/w), sodium bicarbonate (about 66.1% w/w), a fragrance (about 1% w/w), shea butter (about 0.5% w/w), and a surfactant (about 0.3% w/w).

Aspect 18. The rapidly disintegrating buoyant composition of Aspect 1 comprising an aluminum sulfate (about 19.5% w/w), citric acid (about 12.6% w/w), sodium bicarbonate (about 66.1% w/w), a fragrance (about 1% w/w), shea butter (about 0.5% w/w), and tomadol 1-9 (0.3% w/w).

Aspect 19. The rapidly disintegrating buoyant composition of Aspect 1 comprising an aluminum sulfate (about 19.5% w/w), citric acid (about 59.3% w/w), sodium bicarbonate (about 18.1% w/w), a fragrance (about 1% w/w), shea butter (about 0.5% w/w), and a surfactant (about 0.3% w/w).

Aspect 20. The rapidly disintegrating buoyant composition of Aspect 1 comprising an aluminum sulfate (about 19.5% w/w), citric acid (about 59.3% w/w), sodium bicarbonate (about 18.1% w/w), a fragrance (about 1% w/w), shea butter (about 0.5% w/w), and tomadol 1-9 (0.3% w/w).

Aspect 21. The rapidly disintegrating buoyant composition of Aspect 1 comprising copper sulfate (23.7% w/w), citric acid (about 33.4% w/w), sodium bicarbonate (about 41.7% w/w), a fragrance (about 0.8% w/w), and shea butter (about 0.4% w/w).

Aspect 22. The rapidly disintegrating buoyant composition of Aspect 1 comprising citric acid (about 73.9% w/w), sodium bicarbonate (about 24.4% w/w), a fragrance (about 0.9% w/w), shea butter (about 0.5% w/w), and a surfactant (about 0.3% w/w).

Aspect 23. The rapidly disintegrating buoyant composition of Aspect 1 comprising citric acid (about 15.7% w/w), sodium bicarbonate (about 82.6% w/w), a fragrance (about 0.9% w/w), shea butter (about 0.5% w/w), and a surfactant (about 0.3% w/w).

Aspect 24. The rapidly disintegrating buoyant composition of Aspect 1 comprising citric acid (22.8% w/w), sodium carbonate (75.5% w/w), a fragrance (0.9% w/w), shea butter (0.5% w/w), and a surfactant (0.3% w/w).

Aspect 25. A process for preparing a rapidly disintegrating buoyant composition of any one of Aspects 1-15 and Aspects 16-24, which comprises: (i) mixing the stated ingredients a)-d) to obtain a mixed composition, (ii) spraying the mixed composition with a solution to obtain a moistened solid mixture, (iii) blending the moistened solid mixture to obtain a mixed solid mixture, (iv) adding a portion of the mixed solid mixture to a mold to obtain a molded, mixed solid mixture: (v) applying a pressure of about 60 psi to about 500 psi to the molded, mixed solid mixture to obtain a shaped composition; and (iv) drying the shaped composition to obtain a shaped rapidly dissolving buoyant composition.

Aspect 26. A rapidly disintegrating buoyant composition prepared by the process of Aspect 25.

Aspect 27. A method for treating a recirculating water system, which comprises: adding any one of the rapidly disintegrating buoyant compositions of Aspects 1-14, 16-24, and 26 to the water of the recirculating water system.

### Utility of Formed Compositions

Compositions disclosed herein deliver unit doses to large volumes of water while floating, allowing the rapid dissolution of chemicals without damage to pool surfaces.

Compositions (e.g., tablets) disclosed herein allow for dissolution optimization preventing insoluble or undissolved material from settling to the bottom of the pool where it can damage pool surfaces.

A surfactant helped the tablet remain afloat as well as creating a more aesthetically pleasing fizz.

Not to be limited by theory, it is believed that composition (e.g., tablet) buoyancy relates to the formed composition porosity, thereby allowing water to penetrate into the formed composition to generate carbon dioxide internally. Too much hydrophobic material (e.g., skin oil and/or surfactant) may fill pores or channels of the formed composition, which may cause the formed composition (e.g., tablet) to sink. A hydrophobic material (e.g., a skin oil and/or a surfactant) helps wet the surface(s) of the formed composition, generating more gas and allowing water to wick into the formed composition (e.g., tablet) more readily, which generates more gas and helps keep the tablet afloat, as well as resulting in rapid dissolution.

The compositions disclosed herein have the advantage of rapid dissolution without contacting a pool surface, and thus, no damage to a pool surface occurs. Further, the effervescence, while visually compelling, creates agitation/fragmentation which dissolves the compositions in several minutes with the gas generation providing the buoyancy to keep the tablet afloat. The technology is compatible amongst itself, does not require application packaging and will not negatively impact pool chemistry.

### CITED INFORMATION

U.S. Pat. No. 3,232,869 A, Method of purifying aqueous liquid, issued on Feb. 1, 1966 to Andrew J. Gard (“Gard”).
U.S. Pat. No. 4,389,318 A, Rapidly dissolving mixtures containing trichloroisocyanuric acid, issued on Jun. 21, 1983 to John A. Wojtowicz of the Olin Corporation (“Wojtowicz”).
U.S. Pat. No. 6,852,238 B2, Layered tablet water treatment compositions and method of use, issued on Feb. 8, 2005 to Thomas V. Connelly of Steller Technology Company (“Connelly”).
U.S. Pat. No. 9,815,719 B2, Water treatment system, issued on Nov. 14, 2017 to Curtis Sayre et al. of Bio-Lab, Inc. (“Sayre”).
U.S. Pat. No. 9,839,212 B2, Multicomponent and multilayer compacted tablets, issued on Dec. 12, 2017 to Yeoman et al. of Bio-Lab, Inc. (“Yeoman”).

This application claims priority to U.S. Provisional Patent Application No. 63/293,275, filed on 23 Dec. 2021, the disclosure of which is hereby incorporated by reference herein in its entirety along with the subject matter of all information cited herein. If there is a difference between the meaning of subject matter incorporated by reference and the meaning of subject matter disclosed herein, the meaning of the subject matter disclosed herein will control.

1. A rapidly disintegrating buoyant composition, comprising: a) an active agent, b) at least about 15% w/w of an organic acid, c) at least about 10% w/w of a carbonate, and d) about 1% w/w to about 30% w/w of a skin oil, a fragrance, a surfactant, an adjuvant, or a combination thereof.

2. The rapidly disintegrating buoyant composition of claim 1, wherein the rapidly disintegrating buoyant composition has an equivalent ratio of the organic acid to the carbonate of from about 0.3 to about 1.1.

3. The rapidly disintegrating buoyant composition of claim 1, wherein the rapidly disintegrating buoyant composition has an equivalent ratio of the organic acid to the carbonate of from about 0.6 to about 1.1.

4. The rapidly disintegrating buoyant composition of claim 1, wherein active agent comprises a clarifier, an algicide, a phosphate removal composition, a surfactant, an enzyme, a purifying agent, a scale reducing agent, a flocculant, a chelating agent, alkalinity stabilizing agent, non-chlorine oxidant, a fragrance, a dye, a water stabilizer/conditioner, a calcium hardness adjusting agent, a buffering agent, a cyanuric acid removal agent, a chlorine and/or chloramine reducing agent, or a combination thereof.

5. The rapidly disintegrating buoyant composition of claim 1, wherein the organic acid comprises aconitic acid, adipic acid, ascorbic acid, benzoic acid, citric acid, gluconic acid, glutaric acid, glyceric acid, lactic acid, malic acid, malonic acid, succinic acid, tartaric acid, or a combination thereof.

6. The rapidly disintegrating buoyant composition of claim 1, wherein the organic acid is present in an amount of from about 20% w/w to about 75% w/w.

7. The rapidly disintegrating buoyant composition of claim 1, wherein the carbonate comprises a bicarbonate, a carbonate, a sesquicarbonate, or a combination thereof.

8. The rapidly disintegrating buoyant composition of claim 1, wherein the carbonate is present in an amount of from about 10% w/w to about 75% w/w.

9. The rapidly disintegrating buoyant composition of claim 1, wherein the skin oil comprises an avocado butter, an avocado oil, an almond butter, an almond oil, an olive butter, an olive oil, a shea butter, a shea oil, a cocoa butter, a cocoa oil, a coconut butter, a coconut oil, or a combination thereof.

10. The rapidly disintegrating buoyant composition of claim 1, wherein the skin oil ranges from about 1% w/w to about 5% w/w.

11. The rapidly disintegrating buoyant composition of claim 1, wherein the fragrance ranges from about 1% w/w to about 5% w/w.

12. The rapidly disintegrating buoyant composition of claim 1, wherein the surfactant comprises a non-ionic surfactant, a cationic surfactant, an anionic surfactant, a zwitterionic surfactant, an N-oxide surfactant, or a combination thereof.

13. The rapidly disintegrating buoyant composition of claim 1, wherein the surfactant ranges of from about 1% w/w to about 5% w/w.

14. The rapidly disintegrating buoyant composition of claim 1, wherein the adjuvant comprises sodium gluconate, edetic acid, a salt of edetic acid, a N-(1-carboxyethyl)-iminodiacetic acid, a salt of N-(1-carboxyethyl)-iminodiacetic acid, a salt of aconitic acid, a salt of adipic acid, a salt of ascorbic acid, a salt of benzoic acid, a salt of citric acid, a salt of gluconic acid, a salt of glutaric acid, a salt of glyceric acid, a salt of lactic acid, a salt of malic acid, a salt of malonic acid, a salt of succinic acid, a salt of tartaric acid, or a combination thereof.

15. The rapidly disintegrating buoyant composition of claim 1, the adjuvant ranges from about 0% w/w to about 30% w/w.

16. The rapidly disintegrating buoyant composition of claim 1 comprising an aluminum sulfate in an amount of about 19.5% w/w, citric acid in an amount of from about 23% w/w to about 47% w/w, sodium bicarbonate in an amount of from about 32% w/w to about 56% w/w, a fragrance in an amount of about 0.9% w/w, shea butter in an amount of about 0.5% w/w, and a surfactant in an amount of about 0.3% w/w, wherein the equivalent ratio of citric acid to carbonate ranges from about 0.3 to about 1.1.

17. The rapidly disintegrating buoyant composition of claim 1 comprising:
(a) an aluminum sulfate in an amount of about 19.5% w/w, citric acid in an amount of about 12.6% w/w, sodium bicarbonate in an amount of about 66.1% w/w, a fragrance in an amount of about 1% w/w), shea butter (about 0.5% w/w), and a surfactant (about 0.3% w/w);
(b) an aluminum sulfate in an amount of about 19.5% w/w, citric acid in an amount of about 59.3% w/w, sodium bicarbonate in an amount of about 18.1% w/w, a fragrance in an amount of about 1% w/w, shea butter in an amount of about 0.5% w/w, and a surfactant (about 0.3% w/w);
(c) copper sulfate in amount of about 23.7% w/w, citric acid in an amount of about 33.4% w/w, sodium bicarbonate in an amount of about 41.7% w/w, a fragrance in an amount of about 0.8% w/w, and shea butter in an amount of about 0.4% w/w;
(d) citric acid in an amount of about 73.9% w/w, sodium bicarbonate in an amount of about 24.4% w/w, a fragrance in an amount of about 0.9% w/w, shea butter in an amount of about 0.5% w/w, and a surfactant in an amount of about 0.3% w/w;
(e) citric acid in an amount of about 15.7% w/w, sodium bicarbonate in an amount of about 82.6% w/w, a fragrance in an amount of about 0.9% w/w, shea butter in an amount of about 0.5% w/w, and a surfactant in an amount of about 0.3% w/w; or
(f) citric acid in an amount of 22.8% w/w, sodium carbonate in an amount of 75.5% w/w, a fragrance in an amount of 0.9% w/w, shea butter in an amount of 0.5% w/w, and a surfactant in an amount of 0.3% w/w;
wherein the surfactant optionally comprises tomadol 1-9.

18-24. (canceled)

25. A process for preparing a rapidly disintegrating buoyant composition of claim 1, which comprises:
(i) mixing the stated ingredients a)-d) to obtain a mixed composition,
(ii) spraying the mixed composition with a solution to obtain a moistened solid mixture,
(iii) blending the moistened solid mixture to obtain a mixed solid mixture,
(iv) adding a portion of the mixed solid mixture to a mold to obtain a molded, mixed solid mixture;
(v) applying a pressure of about 60 psi to about 500 psi to the molded, mixed solid mixture to obtain a shaped composition; and
(iv) drying the shaped composition to obtain a shaped rapidly dissolving buoyant composition.

26. The shaped composition prepared by the process of claim 25.

27. A method for treating a recirculating water system, which comprises: adding the rapidly disintegrating buoyant compositions of claim 1 to the water of the recirculating water system.
